(19) United States
(12) Patent Application Publication
(10) Pub. No.: US 2015/0066079 A1
(43) Pub. Date: Mar. 5, 2015

(54) SUTURE TAPE WITH EXTERIOR SUTURE STRANDS

(71) Applicant: Arthrex, Inc., Naples, FL (US)

(72) Inventor: John W. Schmieding, Naples, FL (US)

(21) Appl. No.: 14/469,020

(22) Filed: Aug. 26, 2014

Related U.S. Application Data
(60) Provisional application No. 61/870,432, filed on Aug. 27, 2013.

(51) Int. Cl. A61B 17/04

(52) U.S. Cl. CPC A61B 17/0401; A61B 17/04; A61B 2017/0464
USPC 606/228; 606/232

(57) ABSTRACT

Tape constructs for fixation of soft tissue to bone, or of soft tissue to soft tissue. The tape construct includes a thin tape material with at least one thread positioned on the exterior of the thin tape material. Preferably, two threads are provided only on the exterior of the thin tape material, the threads extending in a direction about longitudinal with the thin tape material and abutting the edges or rims of the tape. The two exterior threads act like a parachute for compression of tissue and also allow the tape to fold easily to reduce the diameter of the tape construct for tissue passing and instrument passing. The construct is a simple, collapsible, flexible, parachute-like structure that allows easy threading and passing through instruments.

### CROSS-REFERENCE TO RELATED APPLICATION

This application claims the benefit of U.S. Provisional Application No. 61/870,432 filed Aug. 27, 2013, the entire disclosure of which is incorporated by reference in its entirety herein.

### FIELD OF THE INVENTION

The present invention relates to the field of surgery and, more particularly, to improved sutures and methods of tissue fixation.

### BACKGROUND OF THE INVENTION

Sutures have been known throughout the history of surgery. Recently, the variety of materials used to close wounds and attach soft tissue to bone (or soft tissue to soft tissue) has included a wide selection of synthetic compositions such as ultrahigh molecular weight polyethylene (UHMWPE), or the FiberWire® suture described in U.S. Pat. No. 6,716,234, or the FiberTape® suture tape described in U.S. Pat. No. 7,892,256, the disclosures of both of which are incorporated by reference in their entirety herewith.

FiberTape® is a suture tape with an ultra-high strength, about 2 mm wide, and with a structure similar to that of the FiberWire® suture. The tape provides broad compression and increased tissue cut-through resistance making it a preferred choice for knotless rotator cuff repairs or for high demand applications like AC joint reconstruction and other areas where tissue pull-through may be a concern.

There is a need for an improved suture tape construct with could fold easily to provide a construct with a reduced diameter for tissue passing and instrument passing. Also needed are improved methods of suture manipulation with decreased suture tear, as well as methods of maximizing benefits of tissue cut-through resistance with minimal and efficient use of material.

### SUMMARY OF THE INVENTION

The present invention provides tape constructs for fixation of soft tissue to bone, or of soft tissue to soft tissue. The tape construct includes a thin tape material with at least one thread positioned on the exterior of the thin tape material. Preferably, two threads are provided only on the exterior of the thin tape material, the threads extending in a direction about longitudinal with the thin tape material and abutting the edges of the tape. In this manner, the two exterior threads act like a parachute for compression of tissue and also allow the tape to fold easily to reduce the diameter of the tape construct for tissue passing and instrument passing. The construct is a simple, collapsible, flexible, parachute-like structure that allows easy threading and passing through instruments.

These and other features and advantages of the present invention will become apparent from the following description of the invention that is provided in connection with the accompanying drawings and illustrated embodiments of the invention.

### BRIEF DESCRIPTION OF THE DRAWINGS

FIGS. 1-4 illustrate various tape constructs in accordance with four exemplary embodiments of the present invention.

FIGS. 5-7 illustrate the tape construct of FIG. 1 employed for fixation of tissue to bone, and in accordance with three exemplary embodiments of the present invention.

### DETAILED DESCRIPTION OF THE PREFERRED EMBODIMENTS

The present invention provides methods and constructs for fixation of soft tissue to bone (or of soft tissue to soft tissue) which protect the soft tissue and allow improved tissue passing and instrument passing.

Instead of using a central thread or network through the FiberTape® suture tape, the improved tape construct of the present invention is provided with two threads only on the exterior of a thin tape material (each thread on each longitudinal side/edge of the thin tape material). This aspect is different from the currently manufactured tapes known in the art which typically have a flexible strand (for example, a FiberWire® suture) spliced through the tape, the tape being woven over a central suture strand. The tape construct of the present invention is provided with two exterior strands that act like a parachute for compression of tissue and also allow the construct to fold easier to reduce diameter for tissue passing and instrument passing.

The tape construct may be also manufactured with less material given the fact that the outside strands provide the longitudinal strength of the construct.

The tape construct may be employed in various knotless and knotted tissue repairs and in conjunction with various fixation devices, for example, with a double eyelet PushLock®/suture anchor in general, to allow separation of the outside strands from which the tape is woven between.

The present invention provides a tape construct formed of a thin tape material with at least one thread positioned on the exterior of the thin tape material. Preferably, a plurality of threads (for example, two or more threads) are provided only on the exterior of the thin tape material, the threads extending in a direction about parallel to a longitudinal axis of the thin tape material, and being positioned along the sides and/or edges of the tape. In this manner, the two exterior threads act like a parachute for compression of tissue and allow the tape to fold easier to reduce the diameter of the tape construct for tissue passing and instrument passing.

The present invention also provides a suture tape construct formed of a tape section provided as a flat braid, the flat braid being supported by at least one flexible strand (for example, two suture strands or threads) extending only on the exterior of the suture tape, for example, along the longitudinal sides and/or edges of the flat braid. The at least one flexible strand may extend along portions of the flat braid, for example, along portions of one or more sides and/or edges of the flat braid (for example, the exterior edges of the flat braid), or only along one exterior side and/or edge of the flat braid, or along an exterior side and/or edge of the flat braid and on portions of another exterior edge and/or side of the flat braid. If two or more flexible strands are employed, the flexible strands may be provided about parallel or non-parallel to each other. The strands may be also provided so that one of the strands extends on one side of the flat braid wherein at least another strand extends on another side of the flat braid.

The flexible strands (sutures or cords) may be attached to the suture tape by simply being inserted along the edges/sides of the tape, or by just being tied to the tape. Alternatively, the flexible strands (sutures) could be attached to the tape by any other means of attachment known in the art. In an exemplary embodiment, the flexible strands (sutures or cords) are weaved into the tape during the weaving process. The flexible strands may be permanently or removably attached to the suture tape. The flexible strands may be made of various materials and in different geometries and with different diameters, to provide the suture tape with the ability to fold and be inserted through various passing instruments to aid in the fixation of tissue.

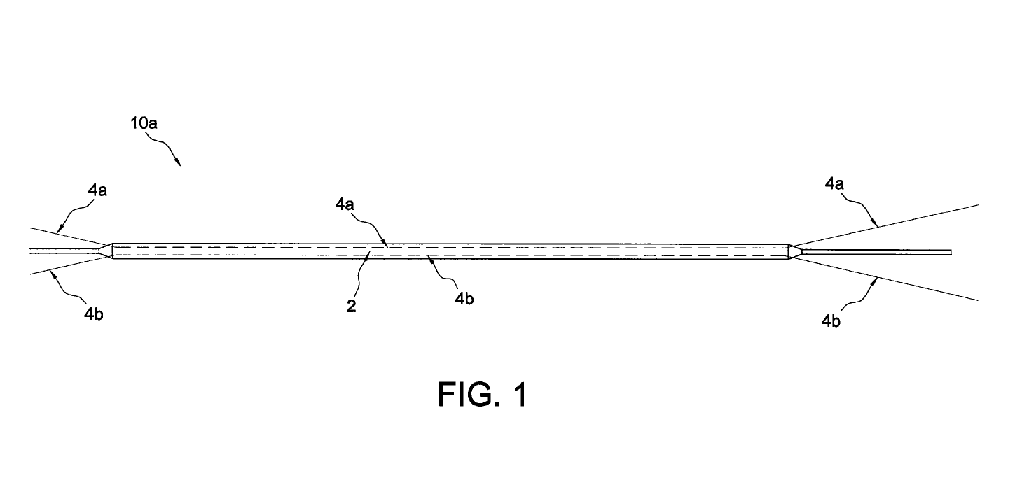

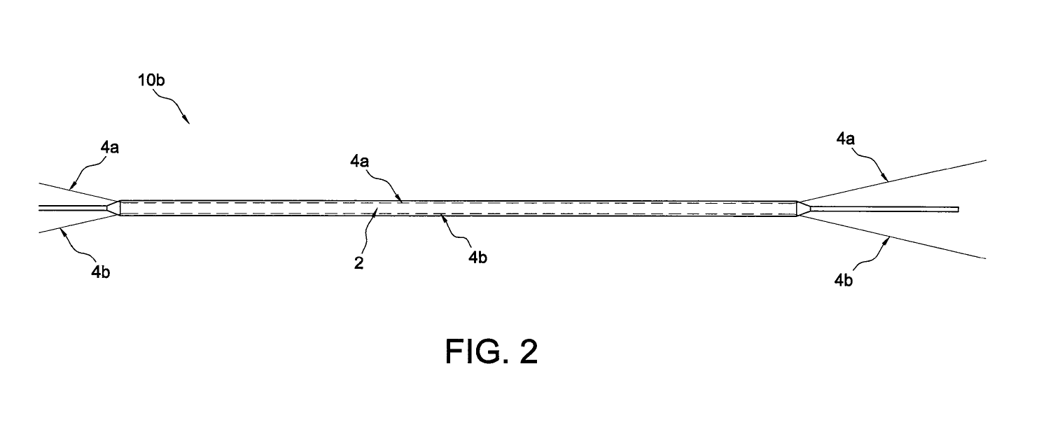

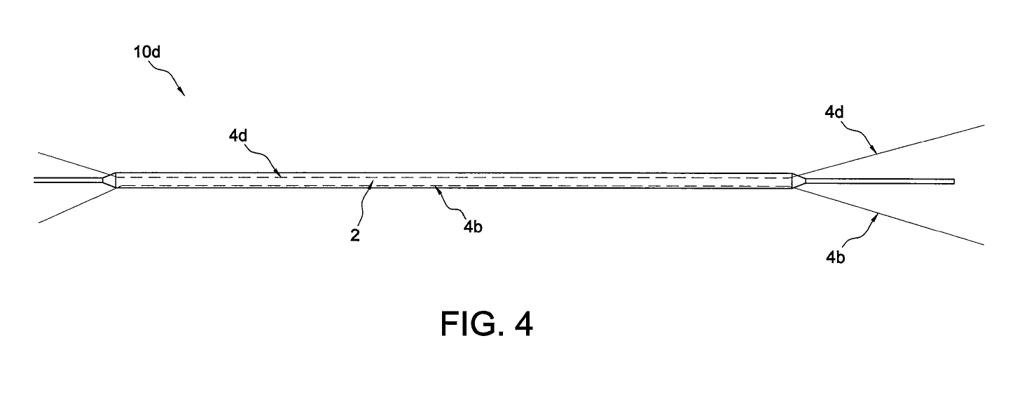

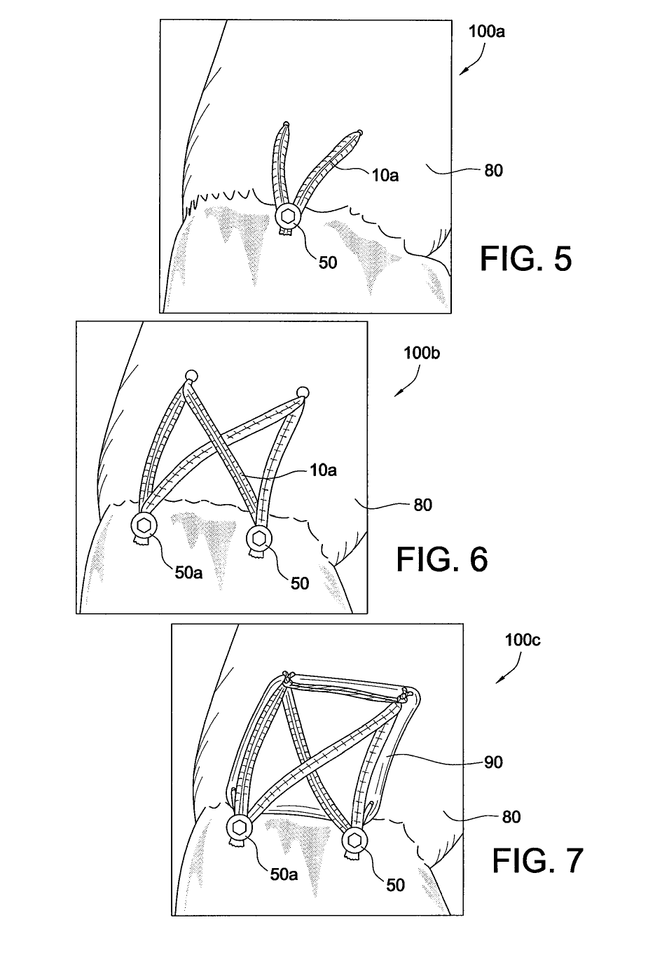

Referring now to the drawings, where like elements are designated by like reference numerals, FIGS. 1-4 illustrate exemplary tape constructs 10a, 10b, 10c, 10d of the present invention. FIGS. 5-7 illustrate exemplary surgical repairs 100a, 100b, 100c with the exemplary tape construct 10a of FIG. 1. Each tape construct 10a, 10b, 10c, 10d is formed of a tape section 2 provided as a thin tape material 2 or a flat braid 2, the tape section 2 being supported by at least one flexible strand 4a, 4b, 4c, 4d (for example, two suture strands or threads) extending only on the exterior of the tape section 2, for example, along only the edges of the tape section 2 (as shown in FIG. 2) or along the sides and edges of the tape section 2 (as shown in FIGS. 1, 3 and 4).

Preferably, the tape construct is formed of tape section 2 and two threads 4a, 4b provided only on the exterior of the tape section 2. Thus, the tape construct of the present invention differs from the FiberTape® suture tape described in U.S. Pat. No. 7,892,256, which has FiberWire® suture spliced through the tape, the tape being woven over a central suture strand. By eliminating the central suture strand and providing two exterior strands 4a, 4b, the construct of the present invention could fold easier than the FiberTape® to reduce diameter for tissue passing and instrument passing. The two exterior strands 4a, 4b of the tape construct also act as a parachute for compression of tissue.

The at least one flexible strand 4a, 4b, 4c, 4d may extend along portions of the tape section 2, for example, along portions of one or more sides/edges of the tape section (such as the exterior edges of the tape), or only along one exterior side (or edge) of the tape, or along an exterior side (or edge) of the tape and on portions of another exterior edge of the tape.

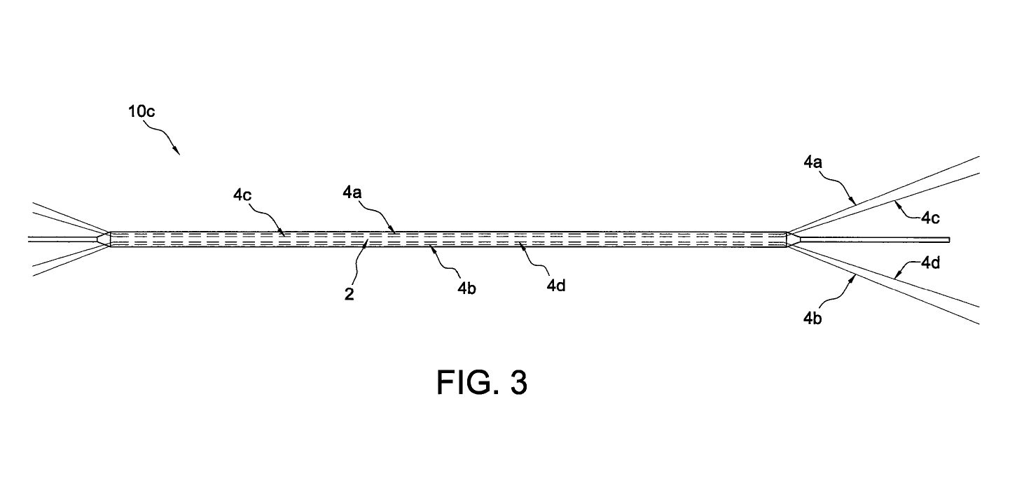

If two or more flexible strands are employed, the flexible strands may be provided about parallel or non-parallel to each other. The strands may be also provided so that one of the strands extends on one side of the tape section wherein at least another strand extends on another side of the tape section. For example, FIG. 3 illustrates an embodiment with four strands, i.e., wherein strands 4a and 4b reside on a first surface (for example, a top surface) of the tape section 2, and strands 4c and 4d reside on a second surface (for example, a bottom surface) of the tape section 2.

The tape section 2 may have compositions and physical and chemical properties similar to those of the flat braid disclosed in U.S. Pat. No. 7,892,256 (the disclosure of which is incorporated in its entirety herewith).

Preferably, the tape section 2 is a thin tape material formed of textile(s) that include all fiber materials which are interlaced and non-interlaced, whether made by weaving, knitting, bonding, laminating, felting, or other processes. All or some of the textile components can be bioabsorbable or resorbable, as known in the art.

The tape section 2 may be also provided as a thin flat braid formed of braided fibers, for example, braided ultrahigh molecular weight polyethylene fiber blended with fibers of one or more long chain synthetic polymers, preferably polyester.

The flexible strands or threads (suture strands) may be provided as cords, preferably braided from suture material strands including UHMWPE fibers blended with fibers of one or more long chain synthetic polymers, most preferably polyester. The UHMWPE fibers impart strength, while the blended fibers, particularly polyester, improve tie down properties.

The flexible strands (sutures) may be attached to the tape section (suture tape) by simply being inserted along the edges of the tape, or by just being tied to the tape. Alternatively, the flexible strands (sutures) could be attached to the tape by any other means of attachment known in the art. The flexible strands may be permanently or removably attached to the suture tape. The flexible strands may be made of various materials and in different geometries and with different diameters, to provide the suture tape with the ability to fold and be inserted through various passing instruments to aid in the fixation of tissue. The flexible strands may be provided right on the edges of the thin tape material, to define the edges or rims of the thin tape material, or may be provided within the body of the thin tape material, i.e., 1-2 mm within the body of the tape and extending along the borders of the tape but spaced from the longitudinal edges/sides of the thin tape material.

If desired, the tape constructs 10a, 10b, 10c, 10d may be provided with one or more transition sections developed at either end of the flat braid. The transition sections are preferably tapered to allow the suture tape to pass easily through openings during surgical procedures. The tape construct 10a, 10b, 10c, 10d may be also provided with materials that have properties to amplify the body's healing response. For example, the tape construct 10a, 10b, 10c, 10d may be provided with a medicinal or therapeutic agent, for example, antiseptics, antibiotics, drugs, pharmaceutical agents, hormones and growth materials (for example, autogenous growth factors such as platelet-rich plasma (PRP), autologous factors, autologous-conditioned plasma (ACP)), among many others. The tape section 2 and/or the flexible strands 4a, 4b, 4c, 4d may be provided with the medicinal or therapeutic agent.

The exemplary tape constructs 10a, 10b, 10c, 10d of the present invention may be employed in various tissue repairs such as knotless rotator cuff repair with the SpeedBridge™ and SpeedFix™ repair techniques, or similar reattachment techniques of soft tissue to bone employing knotless fixation devices for the formation of single, double or multiple row constructs in arthroscopic rotator cuff repairs, or high demand applications like AC joint reconstruction and other areas where tissue pull-through may be a concern.

For example, the SpeedFix™ and SpeedBridge™ techniques, both developed by Arthrex, Inc., use a threaded swivel anchor such as Arthrex SwiveLock® C anchor as disclosed and described in US Publication Nos. 2007/0191849 and 2008/0004659 (the disclosures of all of which being incorporated by reference in their entirety herewith) combined with FiberTape® (disclosed in U.S. Pat. No. 7,892,256) to create a quick and secure SpeedFix™ construct (a knotless single row repair) or a SpeedBridge™ construct (a knotless double row repair) with no knots and very few suture passing steps. By the present invention, the FiberTape® is substituted with the tape construct 10a, 10b, 10c, 10d of the present invention.

In the SpeedBridge™ technique, a swivel anchor (preferably an Arthrex 4.75 mm SwiveLock® C anchor) loaded with one strand of FiberTape® is inserted into a medial bone socket. A FiberLink™ and Scorpion™ shuttle both FiberTape® tails through the rotator cuff simultaneously. Next, one FiberTape® tail from each medial anchor is retrieved and loaded through another SwiveLock® C eyelet. The loaded eyelet is inserted into a prepared lateral bone socket until the anchor body contacts bone, and the tension is adjusted if necessary. The SwiveLock® C driver is rotated in a clockwise direction to complete the insertion. Using an open ended FiberWire® cutter, the FiberTape® tails are cut to complete the technique. By the present invention, the FiberTape® is substituted with the tape construct 10a, 10b, 10c, 10d of the present invention.

FIG. 5 illustrates an exemplary SpeedFix™ repair 100a (knotless single row repair 100a) with tape construct 10a of the present invention, for knotless rotator cuff repair with a knotless fixation device 50 (for example, a SwiveLock® C 50) that secures tape construct 10a over rotator cuff 80.

FIG. 6 illustrates an exemplary SpeedBridge™ repair 100b (knotless double row repair 100b) with tape construct 10a of the present invention, for knotless rotator cuff repair with knotless fixation devices 50, 50a (for example, SwiveLock® anchors 50, 50a) that secure tape construct 10a over rotator cuff 80. The repair requires only two suture passing steps.

FIG. 7 illustrates an exemplary SpeedBridge™ repair 100c (knotless double row repair 100c) with tape construct 10a of the present invention and augmented with a patch 90, for knotless rotator cuff repair with knotless fixation devices 50, 50a (for example, SwiveLock® anchors 50, 50a) that secure tape construct 10a and patch 90 over rotator cuff 80.

Although the invention has been described with reference to a particular application (i.e., shoulder repair), it must be understood that the construct of the present invention has applicability to any type of repairs (any repair in addition to a shoulder repair) and, thus, the invention is not limited by this exemplary-only embodiment.

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all falling within the scope of the invention. Accordingly, the invention is not to be considered as limited by the foregoing description.

What is claimed is:

1. A surgical suture, comprising:
a tape section having a flattened profile and being provided as a flat braid formed of braided strands, the tape section having a length and two longitudinal sides; and
two flexible cords, each of the flexible cord being provided on each of the two longitudinal sides of the tape section.

2. The surgical suture of claim 1, wherein each of the two flexible cords has a length greater than the length of the tape section.

3. The surgical suture of claim 1, wherein each of the two flexible cords is permanently affixed to the longitudinal side of the tape section.

4. The surgical suture of claim 1, wherein the flexible cords include ultrahigh molecular weight polyethylene.

5. The surgical suture of claim 1, wherein the tape section is formed of braided fibers.

6. The surgical suture of claim 5, wherein the tape section is formed of ultrahigh molecular weight polyethylene fibers braided with polyester fibers.

7. The surgical suture of claim 1, wherein the tape section is formed of a material selected from the group consisting of yarns, filaments, fibers, suture strands, textiles, suture tape, polymers, elastic materials, foam and suture-like materials.

8. The surgical suture of claim 1, wherein each of the two flexible cords is stronger than the tape section.

9. A surgical fixation construct consisting essentially of a knotless fixation device and a surgical tape comprising a suture tape provided along a length of two flexible cords, each of the flexible cords defining an edge or rim of the suture tape.

10. The surgical fixation construct of claim 9, wherein the surgical tape passes through at least one eyelet of the knotless fixation device.

11. The surgical fixation construct of claim 10, wherein the at least one eyelet is located at a most distal end of the knotless fixation device.

12. The surgical fixation construct of claim 9, wherein the knotless fixation device is a swivel anchor or a pushlock anchor.

13. The surgical fixation construct of claim 9, wherein the knotless fixation device comprises an anchor body and an anchor tip rotatably attached to the anchor body, and wherein the anchor body is configured to be inserted over the anchor tip for securing the fixation device into bone.

14. The surgical fixation construct of claim 13, wherein the anchor body is a cannulated interference screw.

15. The surgical fixation construct of claim 9, wherein the knotless fixation device is preloaded with the surgical tape.

16. A method of fixation of soft tissue, comprising the steps of:
providing a knotless fixation device having an anchor body and a tip with an eyelet, and a surgical tape construct passed through the eyelet, the surgical tape construct comprising a length of tape material supported by two flexible cords;
passing the surgical tape construct around or through tissue to be fixated; and
fixing the knotless fixation device into bone by inserting the anchor body over the tip so that the surgical tape construct forms a loop, and four limbs of the flexible cords are securely attached to the tip of the knotless fixation device.

17. The method of claim 16, wherein the tip is configured to rotate relative to the anchor body.

18. The method of claim 16, wherein the tip is configured to swivel relative to the anchor body.

19. The method of claim 16, wherein each of the two flexible cords are provided around a longitudinal rim or edge of the length of tape material.

20. A method of fixation of a first tissue to a second tissue, comprising the steps of:
providing a suture construct comprising a length of a flexible material and two flexible cords supporting the flexible material, the flexible cords being provided in a direction parallel to the length of the flexible material and on each longitudinal edge or rim of the flexible material to allow the flexible material to fold easily;
passing the suture construct around the first tissue;
attaching the suture construct to a knotless fixation device by threading the suture construct through an eyelet of the knotless fixation device;
placing the knotless fixation device with the attached suture construct into the second tissue.

21. The method of claim 20, wherein the first tissue is soft tissue and the second tissue is bone.

22. The method of claim 20, wherein the suture construct consisting essentially of suture or suture-like materials.
